(19) United States
(12) Patent Application Publication
(10) Pub. No.: US 2023/0119921 A1
(43) Pub. Date: Apr. 20, 2023

(54) METHODS AND APPARATUS FOR PERFORMING PHYSIOLOGICAL MEASUREMENTS USING DIFFUSE OPTICAL IMAGING

(71) Applicants: Steven M. Ebstein, Newton Centre, MA (US); VOTIS Subdermal Imaging Technologies, Ltd., Jerusalem (IL)

(72) Inventors: Zeev Bomzon, Kiryat Tivon (IL); Steven M. Ebstein, Newton Centre, MA (US); EliyahuShalom Kolet, Jerusalem (IL); Ori Kadosh, Tel-Aviv (IL); Amit Barak, Tel-Aviv (IL); Hagai Barak, Tel-Aviv (IL); Arie Oscar Holtz, Jerusalem (IL); Edan Kenig, Ramat Hasharon (IL)

(21) Appl. No.: 17/970,205

(22) Filed: Oct. 20, 2022

Related U.S. Application Data
(60) Provisional application No. 63/257,745, filed on Oct. 20, 2021.

(51) Int. Cl. A61B 5/145; A61B 5/00

(52) U.S. Cl. CPC A61B 5/145; A61B 5/6833; A61B 2562/166

(57) ABSTRACT

An apparatus for performing diffuse optical imaging of blood circulation in a patient, said apparatus comprising: at least one sensor module comprising at least one optical source and at least one photodetector; an interface electronics module; and means for communicating at least one selected from the group consisting of control signals and measurement data between said sensor module and said interface electronics module; wherein said apparatus further comprises a membrane releasably secured to the skin of the patient, said membrane being configured to releasably secure said at least one sensor module to said membrane such that said at least one sensor module is disposed against the skin of the patient.

### REFERENCE TO PENDING PRIOR PATENT APPLICATION

This patent application claims benefit of pending prior U.S. Provisional Patent Application Ser. No. 63/257,745, filed Oct. 20, 2021 by VOTIS Subdermal Imaging Systems, Ltd. and Zeev Bomzon et al. for DIFFUSE OPTICAL IMAGING SENSORS FOR PHYSIOLOGICAL MEASUREMENTS (Attorney's Docket No. VOTIS-3 PROV).

The above-identified patent application is hereby incorporated herein by reference.

### FIELD OF THE INVENTION

Sensors for performing diffuse optical imaging must be effectively and stably optically coupled to the tissue they are being used to image in order to accurately measure the concentration of the tissue components (e.g., blood). Effectively and stably optically coupling such sensors to tissue requires that the sensors be securely mechanically attached (i.e., secured to) the tissue. When using such sensors for imaging patients (some of whom may have wounds or delicate tissue), the coupling of the sensors must also be sufficiently gentle (and sterile) so as to avoid harming the patient. The present invention comprises the provision and use of novel methods and apparatus for reliably and safely securing such sensors to a patient.

### BACKGROUND OF THE INVENTION

Diffuse optical imaging is an imaging technique for interrogating biological tissues using light in order to image tissue structure and measure concentration of tissue components, e.g., blood and its constituents. Tissue generally has a transmission window in the near infrared (NIR) spectrum. Since scattering of light dominates over absorption of light, NIR light is diffused, and therefore computational methods must be used in order to process the measurements to produce a quantitative result.

One type of diffuse optical imaging uses discrete optical sources (sometimes referred to herein as “discrete sources” or “sources”) and optical detectors, e.g., photodetectors (sometimes referred to herein as “detectors”). By way of example but not limitation, such “sources” and “detectors” may comprise LEDs, laser diodes, and silicon photodiodes. Imaging can be accomplished by transmission of interrogating (i.e., imaging) light, i.e., with the source and detector disposed on opposite sides of the tissue to be imaged, or by reflection of the interrogating (i.e., imaging) light, i.e., with the source and detector disposed on the same side of the tissue. With modern electronics, it is straightforward to design systems that enable robust detection of the scattered NIR light over multiple centimeter (cm) long propagation paths. With reflective geometry, this correlates to measurements corresponding to several centimeter (cm) depths within the tissue being imaged, utilizing inexpensive sources and detectors.

An exemplary light-based imaging system is described by Hielscher et al. in U.S. patent application Ser. No. 16/093,775 for MONITORING TREATMENT OF PERIPHERAL ARTERY DISEASE (PAD) USING DIFFUSE OPTICAL IMAGING (sometimes hereinafter referred to as the “Hielscher patent”), which issued as U.S. Pat. No. 11,439,312, and which patent is hereby incorporated herein by reference in its entirety. The system described in the Hielscher patent is designed to measure the concentrations of, and the changes in concentration of, various tissue components, principally oxy-hemoglobin (HbO₂), deoxy-hemoglobin (Hb), and total hemoglobin (Hbₜₒₜ).

The system described in the Hielscher patent, shown schematically in FIG. 1, generally comprises a plurality of sensor modules 5 each housing a plurality of NIR sources 10 and detectors 15. Sensor modules 5 (sometimes hereinafter referred to as “sensor patches” or “patches”), are each connected by a multi-conductor cable 20 to an interface electronics module 25 which drives NIR sources 10 and measures the signals produced by detectors 15 (e.g., photodetectors). Interface electronics module 25 may be connected to a computer 30 configured to process and store data received from interface electronics module 25, and/or to provide instructions to interface electronics module 25 for driving NIR sources 10 and/or detectors 15.

An exemplary sensor module 5 for use in a diffuse optical imaging system is shown in FIGS. 2 and 3. Sensor module 5 generally comprises four NIR sources 10 (e.g., laser diodes (LDs)) and two detectors 15 (e.g., silicon photodiodes (PDs)). The Hielscher patent describes using a fifteen conductor multi-conductor cable 20 for effecting connection of interface electronics module 25 to each sensor module 5, whereby to permit the transmitting of a variety of analog signals therebetween. With the system of the Hielscher patent, multi-conductor cable 20 comprises eight conductors for the four NIR sources 10 (i.e., LDs), four conductors for the detectors 15 (i.e., PDs), and three conductors for shielding. In fact, where NIR sources 10 comprises LDs, such NIR sources typically comprise three leads for each NIR source for conducting signals relating to drive current, return, and a monitor photodiode to control the power output. In addition, it is possible to use small coaxial cables for each detector 15 in order to reduce shielding requirements. Thus, an alternative multi-conductor cable 20 for use with the system of the Hielscher patent could have four leads for the NIR sources 10, times three, plus four leads for the detectors 15, resulting in 16 leads/conductors, and possibly including one more conductor in order to shield the entire cable (i.e., 17 conductors in total).

In order to produce stable and repeatable measurements, NIR sources 10 and detectors 15 must be fixed in close proximity to the patient's skin (i.e., against the surface of the skin) where the measurement is to be made. In particular, detectors 15 should be in optical contact with the skin of the patient, since an air gap between the detectors and the surface of the skin increases the refractive index discontinuity, thereby reducing the efficiency of the optical coupling to the detector. Any change in the position of detector 15 during use, whether detector 15 is in optical contact with the skin of the patient, or whether there is an air gap between detector 15 and the patient's skin, may cause a change in the measured signal. Since the optical output of NIR sources 10 is restricted in angle, and since all “small package” laser diodes (LDs) (i.e., NIR sources 10) have an air gap, the light output is less sensitive to changes in the position of NIR sources 10 than changes in the position of detectors 15, though such changes can still affect the measured signal.

The weight and stiffness of multi-conductor cable 20 results in consequences that affect the usability of the resulting system. More particularly, the weight and stiffness of multi-conductor cable 20 depends on the number of conductors (i.e., leads) contained within the cable, the size (i.e., gauge) of the conductors, the insulation material and thickness thereof (including, if desired, the presence of shielding), and the jacket material of the outer covering of multi-conductor cable 20 (and the thickness of the same).

For a system such as is described in the Hielscher patent configured with typical choices for the components that are used in the exemplary embodiment discussed above, the resulting multi-conductor cable 20 is typically both heavy and stiff. Specifically, multi-conductor cable 20 is sufficiently heavy (and stiff) that it is challenging to comfortably secure sensor module 5 (i.e., the sensor patch) to the skin of the patient.

In addition, it will also be appreciated that due to the heavy (and stiff) nature of multi-conductor cable 20, relatively small movements of the patient's body can stress the cable sufficiently such that, due to its stiffness, the cable tends to resist the movement of the patient and instead tends to effect movement of sensor module 5 relative to the patient's skin.

Thus, it will be appreciated that the means by which the sensor module (i.e., the sensor “patch”) is secured to the skin of the patient is a critical factor which is often determinative as to whether a diffuse optical imaging system can achieve stable and repeatable measurements. In appreciation of this fact, the Hielscher patent discloses several mechanical means for securing sensor module 5 to the skin of the patient, including using a strap extending around the adjacent anatomy (e.g., around the ankle of the patient) to secure the sensor module to the patient's skin, or a sock-like garment configured to hold sensor module 5 in place on a patient's foot. In practice, such approaches require considerable skill on the part of the clinician in order to produce reliable measurements.

The system described in the Hielscher patent has been shown to be an effective means of providing information that assists a physician in diagnosing and treating peripheral artery disease (PAD), a condition common in a large fraction of diabetics, of whom there are hundreds of millions worldwide. Such a system is thus of great interest for screening and monitoring patients for PAD in order to manage PAD more effectively, whereby to improve patient health and avoid complications resulting from unmonitored/untreated PAD (e.g., amputation).

However, current systems such as that disclosed by the Hielscher patent are not designed for commercial deployment, i.e., deployment in which relatively unskilled clinicians are employed to secure sensor module 5 to the skin of a patient. For diabetic patients, the body part of greatest interest for diagnosing PAD is the foot. However, there are many other parts of the body where diffuse optical imaging has been shown to provide useful diagnostic information about the perfusion of the blood or the concentration of various tissue components.

Thus there exists a need for improved methods and apparatus for securing a sensor module to the skin of a patient in order to perform diffuse optical imaging.

### SUMMARY OF THE INVENTION

The present invention comprises the provision and use of new and improved methods and apparatus for securing sensor modules to the skin of a patient, e.g., for use in diffuse optical imaging. The novel methods and apparatus of the present invention enable relatively unskilled clinicians to quickly and easily secure sensor modules to a patient. The novel methods and apparatus of the present invention also reduce the cost and complexity of effectively deploying useful diagnostic systems, thereby helping to address the need to provide reliable capabilities for providing information that assists a physician in diagnosing and treating PAD and other diseases.

In one form of the invention, there is provided an apparatus for performing diffuse optical imaging of blood circulation in a patient, said apparatus comprising:

at least one sensor module comprising at least one optical source and at least one photodetector;

an interface electronics module; and

means for communicating at least one selected from the group consisting of control signals and measurement data between said sensor module and said interface electronics module;

wherein said apparatus further comprises a membrane releasably secured to the skin of the patient, said membrane being configured to releasably secure said at least one sensor module to said membrane such that said at least one sensor module is disposed against the skin of the patient.

In another form of the invention, there is provided a method for performing diffuse optical imaging of blood circulation in a patient, said method comprising:

providing apparatus comprising:
at least one sensor module comprising at least one optical source and at least one photodetector;
an interface electronics module; and
means for communicating at least one selected from the group consisting of control signals and measurement data between said sensor module and said interface electronics module;
wherein said apparatus further comprises a membrane releasably secured to the skin of the patient, said membrane being configured to releasably secure said at least one sensor module to said membrane such that said at least one sensor module is disposed against the skin of the patient;

securing said membrane to the skin of the patient and securing said sensor module to said membrane; and

actuating the at least one optical source and the at least one photodetector.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a prior art diagnostic system comprising a computer, an interface electronics module, a multi-conductor cable, and a sensor module (i.e., patch) mounted on a foot of a patient according to the Hielscher patent;

FIGS. 2 and 3 are schematic views showing a sensor module according to the system of FIG. 1, including a top perspective view (FIG. 2) and a bottom view (FIG. 3) showing exemplary NIR sources (i.e., LDs) and detectors (i.e., PDs);

FIG. 4 is a schematic view showing a novel diffuse optical imaging system formed in accordance with the present invention;

FIGS. 5 and 6 are schematic views showing a novel apparatus for securing a sensor module against the skin of a patient, wherein the novel apparatus uses spring force to secure the sensor module against the skin of the patient;

FIG. 6A is a schematic view showing another novel apparatus for securing a sensor module against the skin of a patient, wherein the novel apparatus uses spring force to secure the sensor module against the skin of the patient;

FIG. 7 is a schematic view showing an exemplary sensor module;

FIGS. 8-11 are schematic views showing another novel apparatus for securing a sensor module against the skin of a patient, wherein the novel apparatus uses magnetic force to secure the sensor module against the skin of the patient;

FIG. 12 is a schematic view showing another novel apparatus for securing a sensor module against the skin of a patient, wherein the novel apparatus uses magnetic force and spring force to secure the sensor module against the skin of the patient;

FIG. 13 is a schematic view of a novel apparatus for splitting NIR sources (i.e., LDs) and detectors (i.e., PDs) into separated portions of the apparatus;

and

FIG. 14 is a schematic view of a novel adhesive which may be used to adhere a sensor module to the skin of a patient.

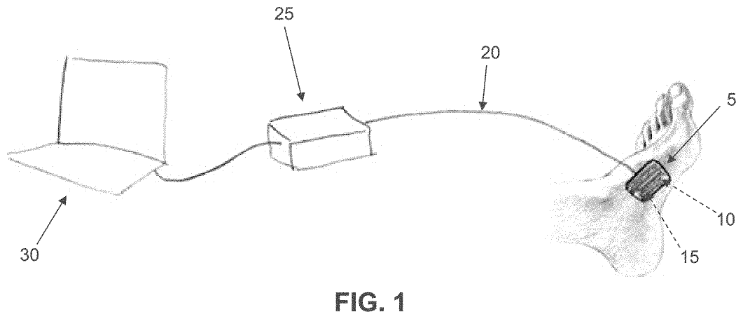

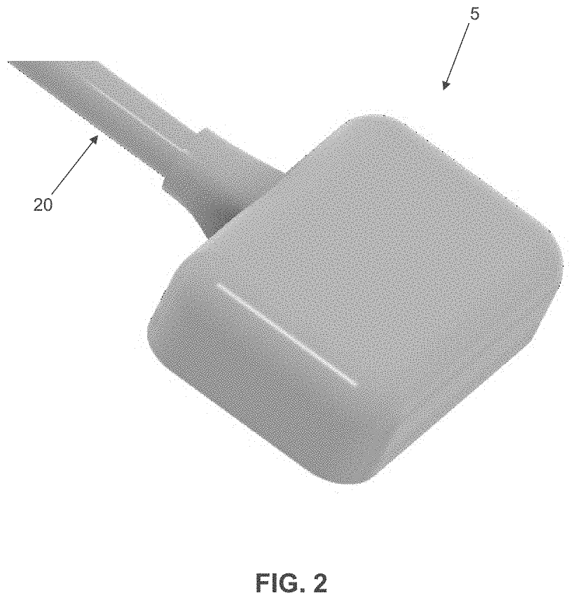

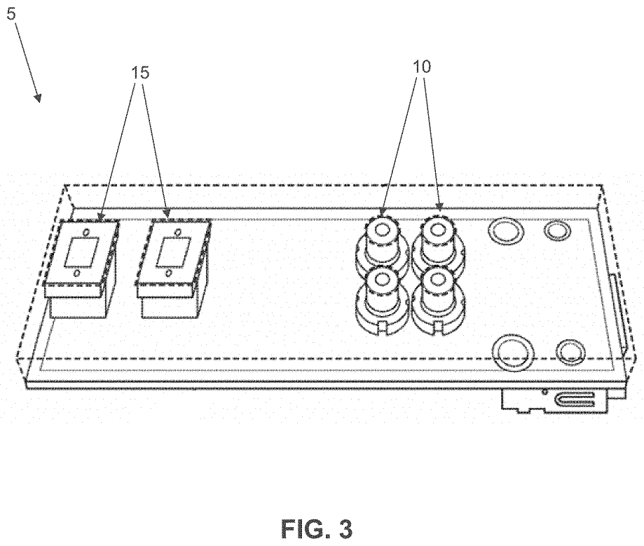

### DETAILED DESCRIPTION OF THE PREFERRED EMBODIMENTS

The present invention comprises the provision and use of new and improved methods and apparatus for securing sensor modules to the skin of a patient, e.g., for use in diffuse optical imaging. The novel methods and apparatus of the present invention enable relatively unskilled clinicians to quickly and easily secure sensor modules to the skin of a patient. The novel methods and apparatus of the present invention also reduce the cost and complexity of effectively deploying useful diagnostic systems, thereby helping to address the need to provide reliable information that assists a physician in diagnosing and treating PAD and other diseases.

Diffuse optical imaging (DOI) is one technique for measuring the concentration of tissue components, e.g., the concentration of oxy- and deoxy-hemoglobin of blood within the human body. DOI can immediately be used to assess oxygen saturation in the tissue. In addition, when coupled with a means of dynamically altering blood flow (e.g., using a pressure cuff to introduce vascular and/or arterial occlusion) the dynamic response of the concentration of oxyhemoglobin and deoxyhemoglobin provides useful diagnostic information for assessing a patient's blood circulation.

As discussed above in the context of the system disclosed in the Hielscher patent, one technique for DOI uses sensor modules mounted to the skin of a patient which contain discrete optical sources (e.g., light emitting diodes (LEDs), laser diodes (LDs), etc.) and discrete optical detectors (e.g., photodiodes (PDs)). In some scenarios it is advantageous to secure a plurality of sensor modules to the patient's skin at different locations, with each sensor module containing a plurality of optical sources and detectors.

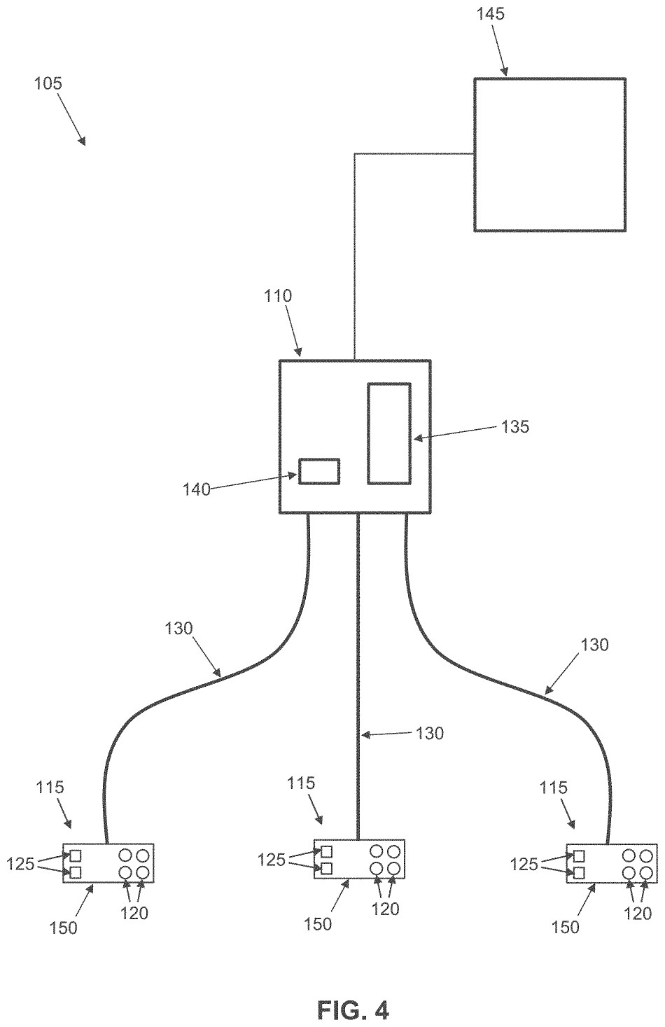

More particularly, and looking now at FIG. 4, there is shown a novel diffuse optical imaging system 105 formed in accordance with the present invention. System 105 generally comprises an interface electronics module 110 having a plurality of sensor modules 115 electrically connected thereto. Each of the plurality of sensor modules 115 comprises a plurality of optical sources 120 for generating an optical signal (e.g., NIR light), and a plurality of detectors 125 (e.g., photodetectors) for detecting light generated by optical sources 120 after the light has passed through the tissue to which sensor module 115 is attached. Each of the plurality of sensor modules 115 is electrically connected to interface electronics module 110 via a multi-conductor cable 130 configured to carry signals that drive optical sources 120 and relay the corresponding signals from detectors 125 back to interface electronics module 110.

In one preferred form of the invention, interface electronics module 110 comprises one or more embedded microcontroller units (MCU) 135 connected to a variety of peripheral integrated circuits (ICs) 140 that manage operation of optical sources 120, detectors 125, and digitization of the measured signals received from detectors 125. Alternatively and/or additionally, if desired, a computer 145 may be connected to the interface electronics module 110 in order to provide the foregoing functionality (and/or such functionality as will be apparent to one of skill in the art in view of the present disclosure).

In a preferred form of the invention, interface electronics module 110 and sensor module(s) 115 each comprise at least one printed circuit board (PCB) (not shown). The sensor module PCB comprises the aforementioned optical sources 120 (e.g., optoelectronic sources which are mounted to the sensor module PCB) and detectors 125 (e.g., photodetectors) as well as appropriate connectors soldered to its PCB, and is contained within a housing 150 constructed of an appropriate material (e.g., a polymer). Interface electronics module 110 typically has a wide variety of electronic components mounted on its PCB(s), as will be apparent to one of skill in the art in view of the present disclosure.

The measurement data produced by sensor module(s) 115 are a function of the parameters of optical sources 120 and detectors 125, as well as the characteristics of the patient such as the concentration of various tissue components, principally oxy-hemoglobin (HbO₂), deoxy-hemoglobin (Hb), and total hemoglobin (Hbₜₒₜ). When sensor module 115 is attached to the patient in a stable manner, stable measurements of the light emitted by optical sources 120 and detected by detectors 125 are obtained which can be used to calculate the parameters of interest. Importantly, accuracy of the measurements obtained using optical imaging system 105 depends on the stability of the optical contact between detectors 125 and the patient's skin.

In addition to promoting accurate measurement of the parameters of interest (e.g., concentration of various tissue components, principally oxy-hemoglobin (HbO₂), deoxy-hemoglobin (Hb), and total hemoglobin (Hbₜₒₜ), etc.), it is important to maintain stability between sensor module(s) 115 and the patient's skin in order to protect the patient, and to ensure good optical coupling between the tissue and detectors 125.

By way of example but not limitation, many patients who suffer from PAD (or who are being screened for PAD) are elderly and/or diabetic. Such patients are susceptible to skin injuries, either due to changes due to age or because poor circulation makes it difficult for the skin to heal in the region where sensor module(s) 115 are to be attached to the skin. Thus, the patient's skin must be protected when sensor module(s) 115 are secured to the skin, and application of sensor module(s) 115 must be made in as sterile a manner as possible. It will be appreciated that the requirement for good optical coupling between detectors 125 and the patient's skin requires detectors 125 to be placed in close contact with the patient's skin (and maintained in position).

Both of these goals (i.e., patient protection and good optical coupling), can be achieved by applying an intermediate layer between sensor module(s) 115 and the patient's skin, e.g., in the form of a gel, a film, etc. By way of example but not limitation, TEGADERM™ is a clear, sterile adhesive film that can be placed on the patient's skin in the region where sensor module(s) 115 is to be secured to the patient. This film strengthens the skin and acts as a sterile barrier between the patient and sensor module(s) 115, allowing the sensor module(s) to be held down against the patient's skin with light pressure. Such pressure can be applied in various ways. By way of example but not limitation, a strap and/or sock-like garment, such as is disclosed in the Hielscher patent, may be used to apply such pressure. Alternatively and/or additionally, tape may be used to adhere sensor module(s) 115 to the patient's skin.

Such attachment means (e.g., a strap, tape, a sock-like garment) all suffer from a common failing. More particularly, it is generally difficult to control the force/pressure that is used to hold sensor module(s) 115 in contact with the skin of the patient. The force/pressure which is applied by a strap or tape is set by the clinician applying the strap or tape, and hence the pressure applied will tend to vary. The pressure applied by a garment such as the sock discussed above will depend on the elasticity of the material out of which the garment is made, as well as the relative sizes of the patient's anatomy (e.g., foot/ankle) and the garment.

Controlling the force/pressure with which sensor module 115 is disposed against the skin of the patient is vitally important because that pressure can affect the concentration of blood underneath the pressure point (and hence the accuracy of the measurement obtained by optical imaging system 105). In addition, varying the blood flow dynamically (e.g., by using a pressure cuff) is a common technique used to assess perfusion (see, for example, the disclosure of the Hielscher patent). The blood concentration at the measurement point (i.e., the point at which sensor module 115 is secured against the skin of the patient) is a function of both force/pressure applied to the patient's tissue by sensor module 115 and, if present, the force/pressure applied to the patient's tissue by a pressure cuff. In order to obtain stable and repeatable measurements, the force/pressure applied by both elements (i.e., sensor module 115 and any pressure cuff used) must be controlled.

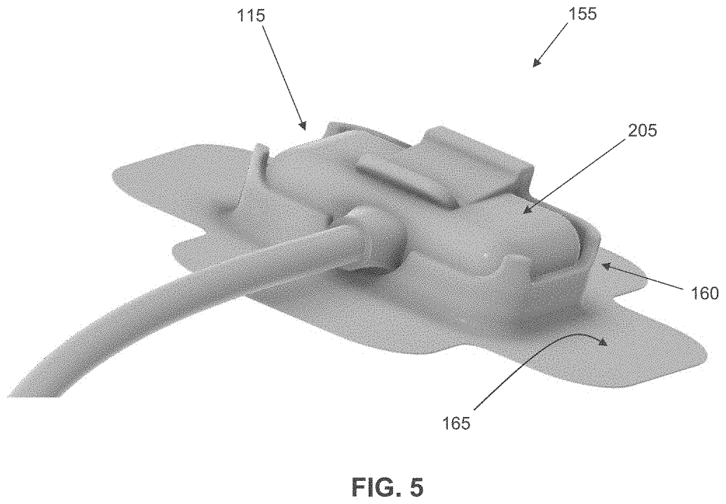

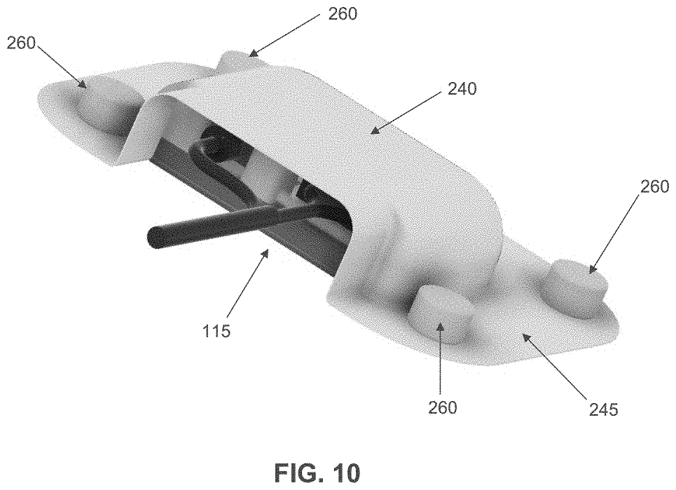

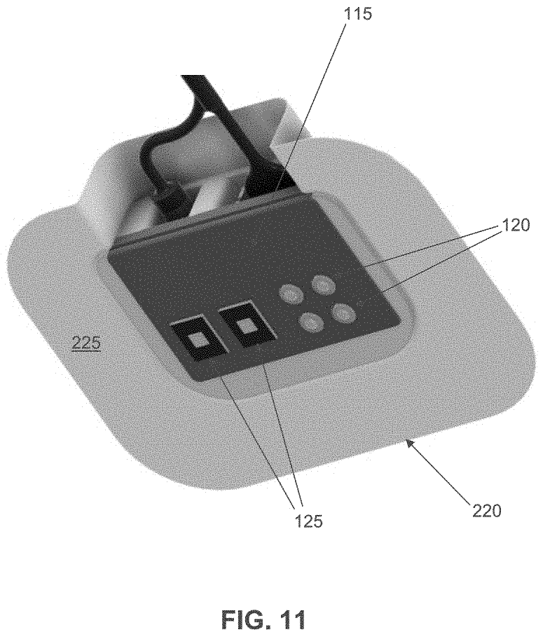

Looking now at FIGS. 5, 6, 6A, 8-12 and 14, there are shown novel apparatus for applying controlled downwardly-directed (i.e., towards the surface of the patient's skin) force/pressure to sensor module 115, whereby to hold sensor module 115 in place against the skin of the patient with the desired degree of force/pressure (i.e., force directed against the skin of the patient). Each novel embodiment of the novel apparatus of the present invention generally comprises a substantially planar structure (e.g., a plate, a membrane, a film, a tape, an assembly, etc.) that is adhered to the skin of the patient so as to permit the securing of a sensor module to the skin of the patient, as will hereinafter be discussed in further detail.

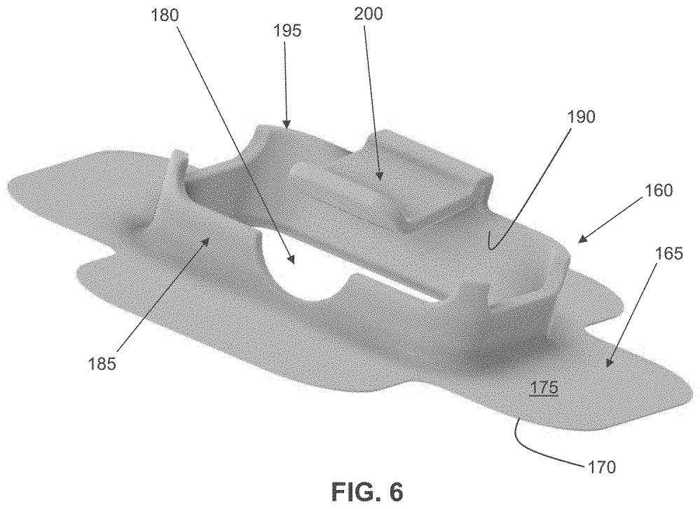

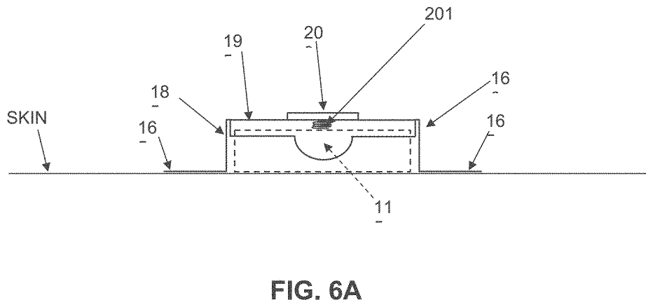

More particularly, and looking now at FIGS. 5 and 6, there is shown a novel clip assembly 155 formed in accordance with the present invention. Clip assembly 155 generally comprises a molded (e.g., plastic) housing 160 and a base 165 comprising a lower surface 170, an upper surface 175 and a central opening 180. Housing 160 comprises a wall 185 extending upward from upper surface 175 and about central opening 180, whereby to define a cavity 190. Wall 185 terminates in an upper surface 195 extending about the perimeter of cavity 190. A spring clip 200 is mounted to (or formed integral with) upper surface 195 of wall 185, and extends partially over cavity 190, generally parallel to the plane defined by base 165, whereby to retain a sensor module 115 within cavity 190 with a predetermined degree of downward force directed towards opening 180, as will hereinafter be discussed in further detail.

In one preferred form of the invention, base 165 comprises a thin, plastic, generally planar structure defined by an outer perimeter that complements the anatomy to which base 165 is to be mounted. Base 165 may hereinafter sometimes be referred to as a “membrane” or “plate”, which terminology is intended to include substantially any generally planar structure, whether flexible or rigid, which is used to secure a sensor module to the body of a patient. Base 165 preferably comprises an adhesive applied to lower surface 170 in order to facilitate securing base 165 to the skin of a patient. If desired, the adhesive applied to lower surface 170 may be covered by a protective sheet (not shown), e.g., wax paper, plastic, etc., that is removed by the clinician in order to expose the adhesive just prior to mounting base 165 to the skin of the patient, as will be apparent to one of skill in the art in view of the present disclosure. Central opening 180 may be open to the skin of the patient, however, it should be appreciated that, if desired, an adhesive film (not shown) that is transparent to light emitted at the wavelengths emitted by optical sources 120 may cover central opening 180.

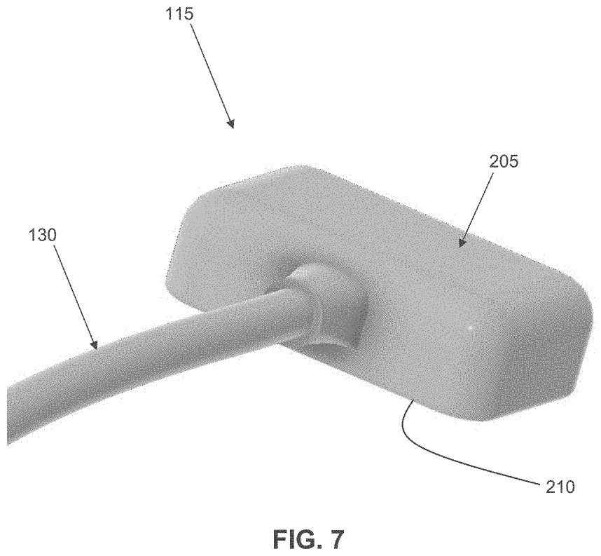

In use, clip assembly 155 is first applied to the region of the patient's skin where a sensor module 115 is to be secured in order to perform a measurement (e.g., diffuse optical imaging). To this end, the clinician removes any protective covering (if provided) that covers the adhesive disposed on lower surface 170 of base 165, and places base 165 against the bare skin of the patient such that the adhesive secures base 165 (and hence, clip assembly 155) to the skin of the patient. It will be appreciated that base 165 is configured to easily bend as necessary in order to mold to the surface of the patient's anatomy. Once clip assembly 155 is mounted to the patient's skin, a sensor module 115 (see FIG. 7) is inserted into cavity 190 of clip assembly 155 by temporarily deflecting spring clip 200 (e.g., moving the free end of spring clip 200 upward) and, once sensor module 115 is disposed within cavity 190, permitting spring clip 200 to return to its initial position (i.e., based on the inherent resiliency of spring clip 200) such that it contacts an upper surface 205 of sensor module 115, and such that a lower surface 210 of sensor module 115 is disposed against the skin of the patient at central opening 180 of clip assembly 155. It will be appreciated that the downwardly-directed force/pressure applied by spring clip 200 to upper surface 205 of sensor module 115 is a function of the mechanical design of spring clip 200, i.e., both the geometry of spring clip 200 and the material out of which spring clip 200 is made (as well as the geometry of sensor module 115 disposed within cavity 190). Thus, it will be appreciated that by controlling these variables, it is possible to provide a combined clip assembly 155 and sensor module 115 which can be mounted to a patient's skin with a predetermined degree of force imposed on sensor module 115 against the patient's skin. This represents a significant advance over the prior art mounting means discussed above, which are partly reliant on the size and shape of the patient's anatomy and therefore cannot be used to mount a sensor module 115 to the skin of a patient with a predetermined amount of downwardly-directed force.

If desired, and looking now at FIG. 6A, at least one spring 201 may be mounted to the underside of spring clip 200 and extend partially into cavity 190 so as to contact the upper surface of a sensor module 115 disposed therein. The downwardly-directed force applied by spring clip 200 to sensor module 115 (and hence, the force applied by sensor module 115 to the skin of the patient) is supplemented by spring 201 which extends between upper surface 205 of sensor module 115 and the underside of spring clip 200. Thus it will be appreciated that the spring force applied by spring 201 to sensor module 115 can be selected in order to set a predetermined downwardly-directed holding force (or pressure) to be applied to sensor module 115.

If desired, spring clip 200 may comprise a screw mechanism (not shown) which can be used to adjust the compression of spring 201, thereby permitting the clinician to adjust the downwardly-directed holding force provided by spring 201.

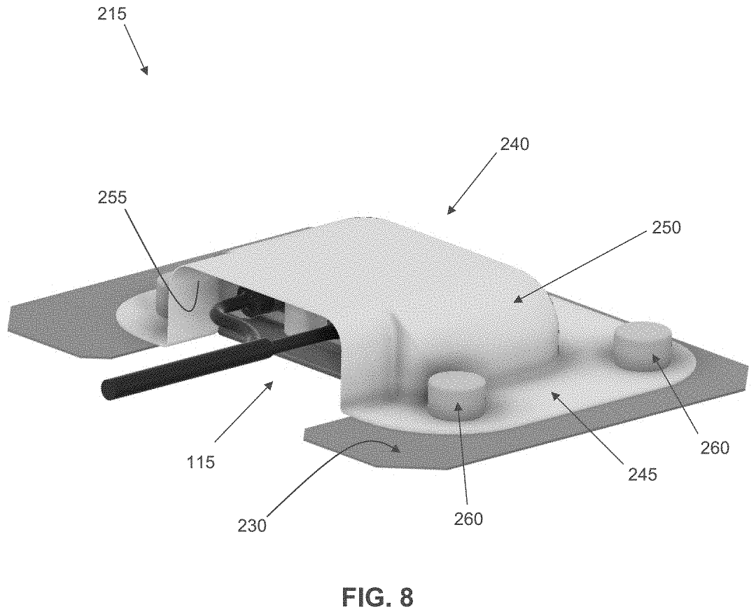

Looking now at FIGS. 8-11, there is shown a novel magnetic clamp assembly 215 formed in accordance with the present invention. Magnetic clamp assembly 215 generally comprises a base 220 comprising a lower surface 225, an upper surface 230 and a central opening 235. Base 220 may hereinafter sometimes be referred to as a “membrane” or “plate”, which terminology is intended to include substantially any generally planar structure, whether flexible or rigid, which is used to secure a sensor module to the body of a patient.

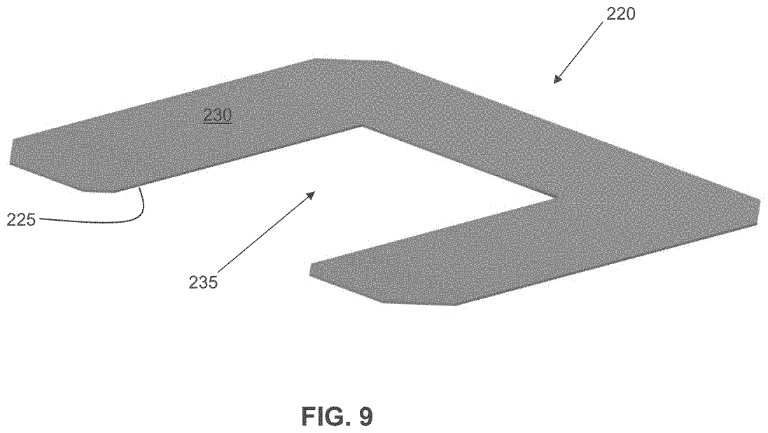

Looking now at FIG. 9, base 220 preferably comprises a thin sheet of ferromagnetic material at upper surface 230, and an adhesive applied to lower surface 225. It will be appreciated that, if desired, central opening 235 may be covered by an adhesive film (not shown) that is transparent to light emitted at the wavelengths emitted by optical sources 120.

A cover 240 is configured to be magnetically mounted to upper surface 230 of base 220, as will hereinafter be discussed in further detail. Cover 240 generally comprises peripheral flange 245 defined by a plane that is coincident with (i.e., parallel to) the plane of base 220 when flange 245 is magnetically mounted thereto, as will hereinafter be discussed in further detail. An outer wall 250 extends generally perpendicular to flange 245 and defines a pocket 255 sized to receive a sensor module 115, as will hereinafter be discussed in further detail. A plurality of magnets 260 are mounted to flange 245 of cover 240 so as to permit flange 245 (and hence, cover 240) to be releasably magnetically mounted to upper surface 230 of base 220, as will hereinafter be discussed in further detail. It will be appreciated that inasmuch as the geometry of pocket 255 is a function of the geometry of cover 240, the geometry of sensor module 115 may be selected so as to complement the geometry of cover 240 and such that, when sensor module 115 is mounted within pocket 255, cover 240 applies a predetermined degree of downwardly-directed force to the upper surface 205 of sensor module 115 (and hence, applies a predetermined degree of downwardly-directed force to sensor module 115 against the skin of the patient).

In one preferred form of the invention, base 220 of magnetic clamp assembly 215 is formed in a generally “U” shape (see, for example, FIG. 9), however, it should be appreciated that base 220 may be formed in other shapes in order to accommodate different regions of the patient's anatomy, as will be apparent to one of skill in the art in view of the present disclosure. If desired, the adhesive applied to lower surface 225 of base 220 may be covered by a protective sheet (not shown), e.g., wax paper, plastic, etc., that is removed by the clinician in order to expose the adhesive just prior to mounting base 220 to the skin of the patient, as will be apparent to one of skill in the art in view of the present disclosure.

In use, base 220 is secured to the skin of the patient by exposing the adhesive disposed on lower surface 225 of base 220 (e.g., by removing any protective covering that covers lower surface 225, if provided) and pressing base 220 against the skin of the patient such that central opening 235 is disposed over the region where a sensor module 115 is to be mounted in order to perform a measurement (e.g., diffuse optical imaging). After base 220 is mounted to the patient's skin, a sensor module 115 is placed over central opening 235 such that lower surface 210 of the sensor module is disposed against the skin of the patient. Cover 240 is then mounted over sensor module 115 and magnetically attached to upper surface 230 of base 220 (i.e., via magnets 260), whereby to hold sensor module 115 within pocket 255 against the skin of the patient with a predetermined amount of force.

The downwardly-directed (i.e., towards the skin of the patient) force/pressure applied to sensor module 115 disposed within pocket 255 of cover 240 will partly depend on the size and magnetic strength of the plurality of magnets 260, as well as the position of magnets 260 on flange 245 relative to base 220. To this end, it should be appreciated that, if desired, magnets 260 may be provided as elements separate from (i.e., not mounted to) flange 245, whereby to permit the clinician to move the magnets around flange 245 in order to mount cover 240 to base 220 such that the desired amount of downwardly-directed force is applied to upper surface 205 of sensor module 115.

However, it will be appreciated that the holding force of the magnets may be difficult to control by varying the magnetic strength and/or position of magnets 260 along flange 245. That is, the magnetic force provided by magnets 260 will decrease with distance if the magnets are spaced away from the ferromagnetic base 220 adhered to the patient, thus compromising the degree of regularity desired in the downwardly-directed force that cover 240 applies to upper surface 205 of the sensor module 115 disposed within pocket 255.

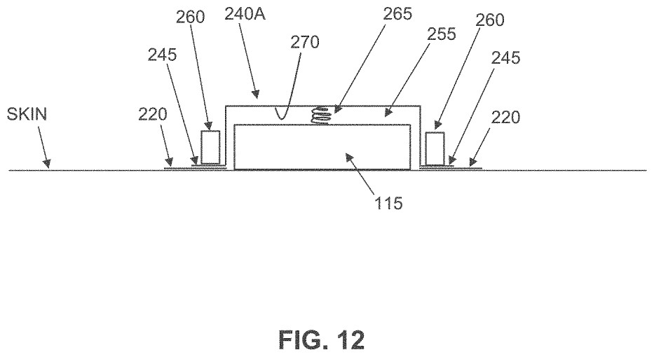

To address this issue, and looking now at FIG. 12, there is shown a cover 240A comprising an alternate means for controlling the downwardly-directed holding force (and hence, pressure) applied to upper surface 205 of sensor module 115. Cover 240A is generally similar to the aforementioned cover 240, however, with this form of the invention, cover 240A comprises at least one spring 265 mounted to the underside 270 of the upper portion of cover 240A and extending partially into pocket 255 so as to contact a sensor module 115 disposed therein. In this form of the invention, magnets 260 are selected so the holding force provided by magnets 260 (i.e., the magnetic force that mounts magnets 260, and hence, cover 240A to flange 245 of base 220) is relatively large, and the base 220 and cover 240A are rigid when they are held together by the at least one magnet 260. The downwardly-directed force applied by cover 240A to sensor module 115 (and hence, the force applied by sensor module 115 to the skin of the patient) is controlled by the at least one spring 265 which extends between upper surface 205 of sensor module 115 and underside 270 of cover 240A. Thus it will be appreciated that the spring force applied by the at least one spring 265 to sensor module 115 can be selected in order to set a predetermined downwardly-directed holding force (or pressure) to be applied to sensor module 115.

If desired, cover 240A may comprise a screw mechanism (not shown) which can be used to adjust the compression of the at least one spring 265, thereby permitting the clinician to adjust the downwardly-directed holding force provided by the at least one spring 265.

It will be appreciated that one issue that affects the stability of securement of a sensor module 115 to the skin of the patient using either clip assembly 155 or magnetic clamp assembly 215 is that the region of the patient's anatomy to which sensor module 115 is attached is generally not flat (e.g., the surface may be rounded, such as where sensor module 115 is to be mounted about the patient's leg or ankle). Thus, the apparatus discussed above (which carefully controls the downwardly-directed pressure applied to sensor module 115 when the attachment surface is flat) may yield different results when clip assembly 155 or magnetic clamp assembly 215 is mounted to a part of the body that is not completely flat. This can be compensated for, in part, by choosing materials for constructing sensor module 115 and/or clip assembly 155 and magnetic clamp assembly 215 that have some flexibility, and thus the ability to conform to the surface of the skin. By way of example but not limitation, a flexible polymer like Polydimethylsiloxane (PDMS) can be selected as the material out of which sensor module 115 and/or clip assembly 155 and magnetic clamp assembly 215 are formed, with a specified flexibility that is a function of the Shore hardness of the selected material. Similarly, if desired, the shape of lower surface 210 of sensor module 115 can be selected to conform to a curve, such that lower surface 210 of sensor module 115 conforms to, and sits against, the underlying tissue (i.e., skin) at various locations on the body.

However, it will also be appreciated that the flexibility of sensor module 115 is also a function of the PCB to which the sensor components are attached. More particularly, a typical FR4 glass epoxy PCB 1.6 mm thick is not very flexible. Other, more flexible PCBs can be used which are less than 0.5 mm thick.

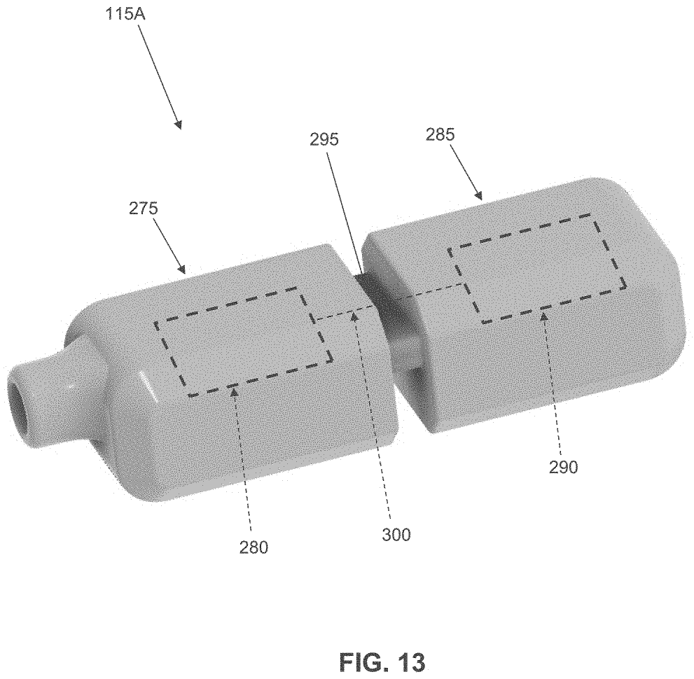

To address this issue, and looking now at FIG. 13, if desired, the PCB housed within sensor module 115 may be split into two or more sections. More particularly, in this form of the invention, there is provided a sensor module 115A comprising a first housing section 275 containing a first PCB 280, and a second housing section 285 containing a second PCB 290. First housing section 275 is mechanically connected to second housing section 285 via a flexible bridge 295. In one preferred form of the invention, first PCB 280 comprises optical sources 120 and second PCB 290 comprises detectors 125. With this form of the invention, it is relatively straightforward to split the PCB (i.e., into first PCB 280 and second PCB 290) such that optical sources 120 are contained in first housing section 275 (mounted to first PCB 280) and detectors 125 are contained in second housing section 285 (mounted to second PCB 290), with one or more wires 300 passing through flexible bridge 295 to bridge the split between first housing section 275 and second housing section 285, whereby to electrically connect first PCB 280 and second PCB 290 to a common multi-conductor cable 130 (not shown). It will be appreciated that “split”-style sensor module 115A can conform perfectly to a surface defined by two intersecting planar regions. In reality, the human body generally comprises curved surfaces, however, such curved surfaces can be well-approximated locally by “split”-style sensor module 115A.

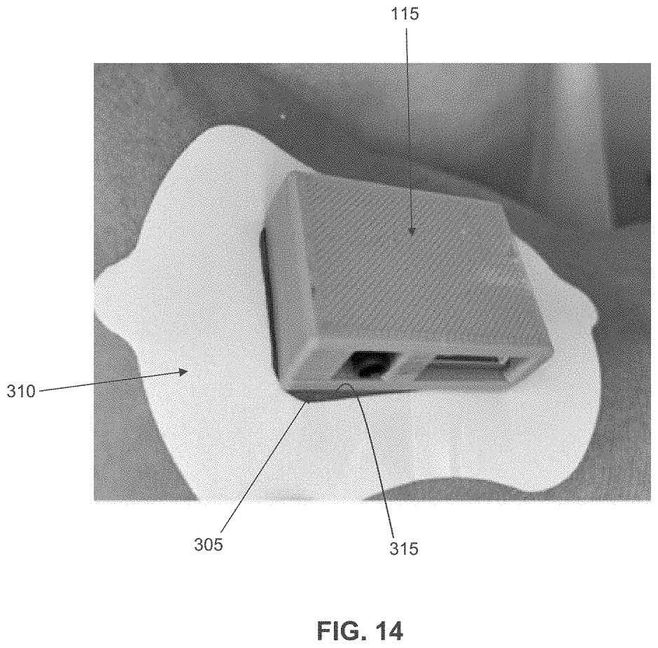

If desired, and looking now at FIG. 14, in another form of the invention, a novel double-sided adhesive film 305 may be used to secure a sensor module 115 to the skin of a patient. With this form of the invention, the upper surface of adhesive film 305 mounts (via an adhesive) directly to lower surface 210 of sensor module 115, and the lower surface of adhesive film 305 mounts directly to the skin of the patient. Film 305 may hereinafter sometimes be referred to as a “membrane” or “plate”, which terminology is intended to include substantially any generally planar structure, whether flexible or rigid, which is used to secure a sensor module to the body of a patient. It will be appreciated that with this form of the invention, adhesive film 305 may be sized to be substantially larger than lower surface 210 of sensor module 115 (i.e., to extend beyond and around the perimeter of sensor module 115 when sensor module 115 is mounted to the skin of the patient). Preferably, a foam layer 310 comprising a central opening 315 for accommodating sensor module 115 may be disposed around sensor module 115, mounted to the upper surface of adhesive film 305. In one preferred form of the invention, double-sided adhesive film 305 comprises TEGADERM™, which film provides a sterile barrier with good optical coupling between sensor module 115 and the skin of the patient. Adding an adhesive layer to the upper surface of adhesive film 305 facing lower surface 210 of sensor module 115 eliminates the need for a mechanism to press the sensor module against the film and the skin.

It will be appreciated that double-sided adhesive film 305 can be manufactured to have the appropriate adhesive to provide the desired holding strength on the upper surface and lower surface of the adhesive film, whereby to tailor each surface of adhesive film 305 so as to provide optimal attachment with the right balance between securing sensor module 115 in position and protecting fragile skin of the patient from stress when the film is attached to the skin (and thereafter removed once the procedure is completed). Foam layer 310 disposed on the upper surface of adhesive film 305 can serve to outline the region where sensor module 115 is to be mounted to the adhesive film, and to help secure sensor module 115 against lateral movement relative to adhesive film 305. If desired, foam layer 310 can comprise a controlled thickness and hardness, and foam layer 310 can be colored (e.g., color-coded), as opposed to the transparent film, e.g., to visually identify the region where sensor module 115 is attached.

It will be appreciated that an additional advantage of securing sensor module 115 to the skin of the patient using double-sided adhesive film 305 is that the nearly flat adhesive film 305 (with or without foam layer 310 disposed on the upper surface thereof) is easily manufactured in roll processes and tends to be less bulky (and less expensive) than systems with molded plastic parts, magnets, steel plates, and springs.

As discussed above, the stability with which a sensor module 115 is secured to the skin of the patient also depends on the multi-conductor cable 130 used to connect sensor module 115 to interface electronics module 110. Even if the local, downwardly-directed force/pressure applied to sensor module 115 is well-defined (e.g., by using an embodiment of the present invention such as the aforementioned clip assembly 155 and/or magnetic clamp assembly 215 and/or double-sided adhesive film 305), undesirable force/pressure may be applied to sensor module 115 by a stiff multi-conductor cable 130 if the cable is not draped in such a way as to minimize its effect on sensor module 115. In addition to draping the multi-conductor cable 130 so that it is parallel to the skin of the patient, such undesirable force/pressure can also be minimized by constructing thinner, more flexible cables using thinner gauge wire and/or more flexible insulation materials around the individual conductors and the overall cable assembly. Stiffness of the shielding material used in multi-conductor cable 130 also has an effect.

An alternative sensor module 115 design could eliminate the need for a multi-conductor cable 130 extending between interface electronics module 110 and sensor module 115. By way of example but not limitation, in another form of the invention, sensor module 115 is constructed so as to contain an internal battery or other power source, and the communication of control signals from interface electronics module 110 to sensor module 115 and/or the communication of measurement data from sensor module 115 to interface electronics module 110 is wireless (e.g., via Bluetooth, Wi-Fi, etc.). Such a “wireless” sensor module 115 eliminates the effect of multi-conductor cable 130 on the pressure applied to the sensor module, which makes it easier to secure sensor module 115 to the skin of a patient and to control the degree of downwardly-directed pressure provided by clip assembly 155, magnetic clamp assembly 215, and/or double-sided adhesive film 305.

### Modifications

Although various exemplary embodiments of the invention have been disclosed, it should be apparent to those skilled in the art that various changes and modifications can be made to the disclosed embodiments without departing from the scope of the invention.

What is claimed is:

1. An apparatus for performing diffuse optical imaging of blood circulation in a patient, said apparatus comprising:
at least one sensor module comprising at least one optical source and at least one photodetector;
an interface electronics module; and
means for communicating at least one selected from the group consisting of control signals and measurement data between said sensor module and said interface electronics module;
wherein said apparatus further comprises a membrane releasably secured to the skin of the patient, said membrane being configured to releasably secure said at least one sensor module to said membrane such that said at least one sensor module is disposed against the skin of the patient.

2. The apparatus of claim 1 further comprising a computer connected to said interface electronics module, said computer comprising a user interface for facilitating operation of said apparatus.

3. The apparatus of claim 1 wherein said membrane comprises a spring clip configured to contact an upper surface of said at least one sensor module, whereby to secure a lower surface of said at least one sensor module against the skin of the patient.

4. The apparatus of claim 3 wherein said spring clip comprises at least one spring extending between the spring clip and the upper surface of said at least one sensor module, whereby to apply a force to said at least one sensor module.

5. The apparatus of claim 4 wherein the force applied by said at least one spring is adjustable.

6. The apparatus of claim 1 wherein said membrane comprises a plate comprising an upper surface and a lower surface, wherein said upper surface comprises a ferromagnetic material and said lower surface is configured to be disposed against the skin of the patient, and further wherein said membrane comprises a cover comprising at least one magnet configured to magnetically mount said cover to said ferromagnetic material of said upper surface of said plate, said cover defining a cavity sized to receive said at least one sensor module, whereby to secure said at least one sensor module against the skin of the patient.

7. The apparatus of claim 6 wherein said cover comprises a top, wherein said top comprises an upper surface and a lower surface, and further wherein said cover comprises at least one spring mounted to said lower surface of said top of said cover and extending into said cavity so as to contact an upper surface of said at least one sensor module, whereby to apply a force to said at least one sensor module.

8. The apparatus of claim 7 wherein the force applied by said at least one spring is adjustable.

9. The apparatus of claim 1 wherein said at least one sensor module comprises a flexible enclosure.

10. The apparatus of claim 1 wherein said at least one sensor module comprises a first housing section flexibly connected to a second housing section, wherein said first housing section comprises a first printed circuit board (PCB) and said second housing section comprises a second PCB.

11. The apparatus of claim 10 wherein said at least one optical source is mounted to said first PCB and said at least one photodetector is mounted to said second PCB.

12. The apparatus of claim 1 wherein said membrane comprises a double-sided adhesive film.

13. The apparatus of claim 8 wherein said double-sided adhesive film comprises an upper surface and a lower surface, wherein a first adhesive is disposed on said upper surface and a second adhesive is disposed on said lower surface, and further wherein said first adhesive and said second adhesive comprise different holding strengths.

14. The apparatus of claim 13 wherein said upper surface of said double-sided adhesive film comprises a first portion comprising an adhesive, and a second portion comprising foam.

15. The apparatus of claim 14 wherein said second portion of said upper surface of said double-sided adhesive film comprises foam having different thickness, material, and/or color than said first portion of said upper surface of said double-sided adhesive film.

16. A method for performing diffuse optical imaging of blood circulation in a patient, said method comprising:
providing apparatus comprising:
at least one sensor module comprising at least one optical source and at least one photodetector;
an interface electronics module; and
means for communicating at least one selected from the group consisting of control signals and measurement data between said sensor module and said interface electronics module;
wherein said apparatus further comprises a membrane releasably secured to the skin of the patient, said membrane being configured to releasably secure said at least one sensor module to said membrane such that said at least one sensor module is disposed against the skin of the patient;
securing said membrane to the skin of the patient and securing said sensor module to said membrane; and
actuating the at least one optical source and the at least one photodetector.

17. The method of claim 16 wherein said apparatus further comprises a computer connected to said interface electronics module, said computer comprising a user interface for facilitating operation of said apparatus.

18. The method of claim 16 wherein said membrane comprises a spring clip configured to contact an upper surface of said at least one sensor module, whereby to secure a lower surface of said at least one sensor module against the skin of the patient.

19. The method of claim 18 wherein said spring clip comprises at least one spring extending between the spring clip and the upper surface of said at least one sensor module, whereby to apply a force to said at least one sensor module.

20. The method of claim 19 wherein the force applied by said at least one spring is adjustable.

21. The method of claim 16 wherein said membrane comprises a plate comprising an upper surface and a lower surface, wherein said upper surface comprises a ferromagnetic material and said lower surface is configured to be disposed against the skin of the patient, and further wherein said membrane comprises a cover comprising at least one magnet configured to magnetically mount said cover to said ferromagnetic material of said upper surface of said plate, said cover defining a cavity sized to receive said at least one sensor module, whereby to secure said at least one sensor module against the skin of the patient.

22. The method of claim 16 wherein said cover comprises a top, wherein said top comprises an upper surface and a lower surface, and further wherein said cover comprises at least one spring mounted to said lower surface of said top of said cover and extending into said cavity so as to contact an upper surface of said at least one sensor module, whereby to apply a force to said at least one sensor module.

23. The method of claim 22 wherein the force applied by said at least one spring is adjustable.

24. The method of claim 16 wherein said at least one sensor module comprises a flexible enclosure.

25. The method of claim 16 wherein said at least one sensor module comprises a first housing section flexibly connected to a second housing section, wherein said first housing section comprises a first printed circuit board (PCB) and said second housing section comprises a second PCB.

26. The method of claim 25 wherein said at least one optical source is mounted to said first PCB and said at least one photodetector is mounted to said second PCB.

27. The method of claim 16 wherein said membrane comprises a double-sided adhesive film.

28. The method of claim 27 wherein said double-sided adhesive film comprises an upper surface and a lower surface, wherein a first adhesive is disposed on said upper surface and a second adhesive is disposed on said lower surface, and further wherein said first adhesive and said second adhesive comprise different holding strengths.

29. The method of claim 28 wherein said upper surface of said double-sided adhesive film comprises a first portion comprising an adhesive, and a second portion comprising foam.

30. The method of claim 29 wherein said second portion of said upper surface of said double-sided adhesive film comprises foam having different thickness, material, and/or color than said first portion of said upper surface of said double-sided adhesive film.
